(19) United States
(12) Patent Application Publication
(10) Pub. No.: US 2010/0204319 A1
(43) Pub. Date: Aug. 12, 2010

(54) TREATMENT FOR BENIGN PROSTATIC HYPERPLASIA

(75) Inventor: David Archibald, Western Australia (AU)

Correspondence Address: J. Rodman Steele;Novak Druce & Quigg LLP, 525 Okeechobee Blvd, Suite 1500, West Palm Beach, FL, 33401, US

(73) Assignee: Summa Development Limited, City Beach, Western Australia (AU)

(21) Appl. No.: 12/446,007

(22) PCT Filed: Oct. 17, 2007
(86) PCT No.: PCT/AU07/01572

§ 371 Date: May 12, 2009

(30) Foreign Application Priority Data
Oct. 17, 2006 (AU) 2006905763

(51) Int. Cl. A61K 31/26; A61P 1/16

(52) U.S. Cl. 514/515

(57) ABSTRACT

A method and composition for the treatment for bladder outlet obstruction, particularly benign prostatic hyperplasia (BPH), which includes administering to the mammal a therapeutically effective amount of a synergistic combination of Capsicum plants and aqueous extract from cruciferous vegetables.

### FIELD OF THE INVENTION

The present invention relates to a method and substance useful for improved bladder function.

More specifically, the present invention is directed towards a method and substance for treatment of benign prostatic hyperplasia (BPH)

### DESCRIPTION OF THE PRIOR ART

In this specification, where a document, act or item of knowledge is referred to or discussed, this reference or discussion is not an admission that the document, act or item of knowledge or any combination thereof was at the priority date: part of common general knowledge, or known to be relevant to an attempt to solve any problem with which this specification is concerned.

Benign prostatic hyperplasia (BPH) is one of the most common benign neoplasms in men and is a result of the prostate increasing in size, which in turn exerts pressure on the urethra.

The incidence of BPH increases with age, with approximately 90% of males having histologically identifiable symptoms at age 85.

One of the most common symptoms of BSA is an incomplete emptying of the bladder which will usually result in an increase in the frequency of urination.

Such symptoms include intermittent urine flow, terminal dribbling, lack of ability to control urine flow, nocturia, and effort required to begin urine flow.

Currently there exists a number of treatments, both medical and surgical, aimed at increasing the peak flow of urine in sufferers. Such treatments include transurethral resection of the prostate (TURP), transurethral incision of the prostate (TJIP), balloon dilation, Finasteride therapy, Alpha blocker therapy or open prostatectomy.

Of the above medical treatments Finasteride is one of the most commonly prescribed medications.

Finasteride is an adrenergic blocker that inhibits 5-α-reductase, which can lead to a reduction in size of the prostate over time, although this is usually measured in months and is not a fast acting medication. Normal urine flow is 25 ml/sec, Finasteride typically raises peak urine flow from 10 ml/sec to 13 ml/sec over some 3 months.

### OBJECT CF THE INVENTION

It is an object of the present invention to provide a method decreasing the size of an enlarged prostate in a mammal.

It is yet a further object of the invention to provide a method of treatment of bladder outlet obstruction.

More specifically, it is an object of the present invention to provide a synergistic combination of sulforaphane and capsaicin for the treatment of bladder outlet obstruction in mammals.

It is yet a further object of the present invention to provide a pharmaceutical preparation including a synergistic combination of sulforaphane and capsaicin for the treatment of bladder outlet obstruction in mammals

More specifically, wherein the cause of the bladder outlet obstruction is benign prostatic hyperplasia.

It is an object of the present invention to overcome, or at least substantially ameliorate. the disadvantages and shortcomings of the prior art.

Other objects and advantages of the present invention will become apparent from the following description, taking in connection with the accompanying drawings, wherein, by way of illustration and example, an embodiment of the present invention is disclosed.

### SUMMARY OF THE INVENTION

According to the present invention, although this should not be seen as limiting the invention in any way, there is provided a method of treatment for bladder outlet obstruction in a mammal, which includes administering to the mammal a therapeutically effective amount of a combination of Capsicum plants and aqueous extract from cruciferous vegetables.

In preference, the bladder outlet obstruction is caused by benign prostatic hyperplasia.

In preference, the combination is a synergistic combination.

In preference, the cruciferous. vegetable is broccoli.

In preference, the broccoli contains sulforaphane.

In preference, the ratio of broccoli extract to Capsicum plants is between 10:1 and 100:1 w/w.

In preference, the ratio of broccoli extract to Capsicum plants is 25:1.

In preference, the capsicum plants are derived from the Capsicum annum species.

In preference, the finely powdered fruits of the capsicum plants are used.

In preference, the fruits contain Capsicum vanilloids.

In preference, the vanilloids are selected from the group consisting of capsaicin and vanillylamine.

A further aspect of the invention resides in a composition for treating bladder outlet obstruction in a mammal, wherein the composition includes a therapeutically effective amount of a synergistic combination of Capsicum plants and an aqueous extract of cruciferous vegetables.

In preference the treatment is applicable to a bladder outlet obstruction which is the result of benign prostatic hyperplasia.

In preference, the cruciferous vegetable is broccoli.

In preference, the broccoli contains sulforaphane.

In preference, the ratio of broccoli extract to Capsicum plants is between 10:1 and 100:1 w/w.

In preference, the ratio of broccoli extract to Capsicum plants is 25:1.

In preference, the capsicum plants are derived from the Capsicum annum species.

In preference, the finely powdered fruits of the capsicum plants are used.

In preference, the fruits contain Capsicum vanilloids.

In preference, the vanilloids are selected from the group consisting of capsaicin and vanillylamine.

### DETAILED DESCRIPTION OF THE INVENTION

Lyophilised broccoli sprouts were obtained from dark grown organic broccoli seeds that had germinated at 25° C. and were 3 days old, freeze dried and ground to a powder. In another instance (Finely ground and sieved sprouts or a commercial preparation of sprouts were cold water extracted overnight and subsequently lyophilized).

Finely powdered Capsicum annum fruits of different varieties (pepper powders were then mixed with the lyophilized broccoli extract in various ratios ranging from 10:1 to 100:1 w/w, broccoli extract: capsicum plant. The embodiment used a 25:1 ratio w/w broccoli powder: capsicum plant

Assays of the broccoli powder shows that the percentage of sulforaphane is 0.1% although more recent production has now taken this value up to 1.9%.

Several early stage studies were conducted in which subjects suffering from bladder outlet obstruction as a result of benign prostatic hyperplasia were treated by swallowing capsules with a synergistic combination of broccoli powder and finely powdered Capsicum annum fruits as earlier specifically described.

### Subject 1

After taking the combination for 3 days the subject's peak urine flow increased dramatically.

### Subject 2

Subject experienced increased urine flow after 5 days, with nocturia being reduced from 5 times per night down to once or twice.

### Subject 3

Experienced increased urine flow rate after only 2 days. PSA (prostate specific antigen) levels fell by 18%

### Subject 4

Increased peak urine flow and nocturia down from 3-4 times per night to once or twice.

### Subject 5

Dramatic increase In peak urine flow after 24 hours of beginning treatment.

Prior to treatment patient showed sighs of elevating levels of prostate specific antigen (PSA). After starting the treatment with combination of broccoli powder and finely powdered Capsicum annum fruits a dramatic decrease in PSA levels were observed. PSA levels halved in 3 months from beginning treatment.

### Subject 6

Experienced positive effect on urine flow, incidence of nocturia also dramatically reduced.

### Subject 7

PSA levels fell from 15.4 to 11.3 within two months of beginning treatment with the combination of broccoli powder and finely powdered Capsicum annum fruits.

A comparison of Finasteride with the present invention is significant_{—} Finasteride may take approximately 3 months to raise urine flow from 10 ml/sec to 13 ml/sec. Contrast this with the present invention in which it has been observed that urine flow has similar effects in 1-3 days, a significant increase in efficacy.

This then for the first time shows the dramatic effect of the synergistic combination of broccoli powder and finely powdered Capsicum annum fruits on benign prostatic hyperplasia and PSA.

Although the invention has been herein shown and described in what is conceived to be the most practical and preferred embodiment, it is recognized that departures can be made within the scope of the Invention, which is not to be limited to the details described herein but it is to be accorded the full scope is of the appended claims so as to embrace any and all equivalent compositions and methods.

1-23. (canceled)

24. A method of treatment for benign prostatic hyperplasia in a mammal, which includes administering to the mammal a therapeutically effective amount of a synergistic combination of Capsicum plants and sulforaphane.

25. The method of claim 24, wherein the sulforaphane is sourced from broccoli extract.

26. The method of claim 25, wherein the ratio of broccoli extract to Capsicum plants is between 10:1 and 100:1 w/w.

27. The method of claim 26, wherein the ratio of broccoli extract to Capsicum plants is 25:1.

28. The method of claim 24, wherein the capsicum plants are derived from the Capsicum annum species.

29. The method of claim 28, wherein the finely powdered fruits of the capsicum plants are used.

30. The method of claim 29, wherein the fruits contain Capsicum vanilloids.

31. The method of claim 30, wherein the vanilloids are selected from the group consisting of capsaicin and vanillylamine.

32. A composition including a therapeutically effective amount of a synergistic combination of Capsicum plants and sulforaphane when used in the treatment of benign prostatic hyperplasia.

33. The composition of claim 32, wherein the sulforaphane is obtained from an aqueous extract of cruciferous vegetables.

34. The composition of claim 33, wherein the cruciferous vegetable is broccoli.

35. The composition of claim 34, the ratio of broccoli extract to Capsicum plants is between 10:1 and 100:1 w/w.

36. The composition of claim 35, the ratio of broccoli extract to Capsicum plants is 25:1.

37. The composition of claim 36, the capsicum plants are derived from the Capsicum annum species.

38. The composition of claim 37, the finely powdered fruits of the capsicum plants are used.

39. The composition of claim 38, the fruits contain Capsicum vanilloids.

40. The composition of claim 39, the vanilloids are selected from the group consisting of capsaicin and vanillylamine.

41. The composition of claim 32, wherein said composition is formulated for the treatment of benign prostatic hyperplasia, substantially as hereinbefore described.
